(19) United States
(12) Patent Application Publication
(10) Pub. No.: US 2021/0123896 A1
(43) Pub. Date: Apr. 29, 2021

(54) Device and Method for Remote Monitoring the State of Grains Stored in All Types of Silos

(71) Applicant: Diego Ruben Barrettino, Lugano (CH)

(72) Inventor: Diego Ruben Barrettino, Lugano (CH)

(21) Appl. No.: 17/083,353

(22) Filed: Oct. 29, 2020

Related U.S. Application Data
(60) Provisional application No. 62/927,514, filed on Oct. 29, 2019.

(51) Int. Cl. G01N 33/02; G01N 33/24

(52) U.S. Cl. CPC G01N 33/02; G01N 2033/245; G01N 33/246

(57) ABSTRACT

An apparatus for remote monitoring the quality and condition of grains stored in all types of silos that can be configured either as a sensing unit or as a communication gateway and a method to use this device in its different configurations for remote monitoring the quality and condition of grains stored in all types of silos.

### CROSS REFERENCE TO RELATED APPLICATIONS

This application is a non-provisional application that claims priority from provisional application Ser. No. 62/927,514 filed on Oct. 29, 2019, the disclosure of which is hereby incorporated herein by reference in its entirety.

### BACKGROUND OF THE INVENTION

The rising population has generated an enormous demand for grains, which has created an ever-increasing interest to increase the grain production and to reduce the losses of grains stored in all types of silos (that is, both traditional silos and silo bags). However, billions of US dollars worldwide are lost every year for the lack of proper monitoring the quality and condition of grains in silos. When these post-harvesting losses are reduced, an important contribution to the efficient use of natural resources and energy can be achieved as well as important economic losses can be avoided.

At present, the grains stored in traditional silos are only monitored by temperature sensors (thermocouples), which are buried in the mass of grains and arranged along resistant cables. The cables must be firmly fixed to the silo structure in order to support the weight and movements of the grain. On the other hand, the grains stored in silo bags, which are usually located in remote locations, are rarely monitored because of the lack of appropriate remote monitoring equipment for silo bags.

Therefore, there is a need for a device and a method based on this device for remote monitoring the state of grains stored in all types of silos.

### SUMMARY OF THE INVENTION

Thus, this invention comprises a device for remote monitoring the quality and condition of grains stored in all types of silos that can be configured either as a sensing unit or as a communication gateway and a method to use this device in its different configurations for remote monitoring the quality and condition of grains stored in all types of silos.

The device configured as sensing unit for silo bags consists of at least four metallic ring electrodes used by the grain moisture sensor, a battery pack, a power management unit, a grain moisture sensor based on electrical impedance or capacitive or microwave measurements, an accelerometer used for the detection of a potential theft or vandalism, an oxygen sensor, a carbon dioxide sensor, an air humidity sensor, an air temperature sensor, a communication module used to communicate with the gateway, a tip in an extreme of the sensing unit, a tool for installation and sealing in the opposite extreme of the sensing unit, a flexible antenna attached to the external side of the tool for installation and sealing, some fins to facilitate the insertion in the silo bag if the grains were very compacted when the silo bag was filled, and some microcontrollers used for handling the sensor data (for example, for data compression and encryption) and for processing the sensor data (for example, for sensor calibration and for pattern recognition). When the device is installed in silo bags, it has the particular feature that can be hidden with a cover patch thanks to the shape of its mechanical structure because only the disk of the tool for installation and sealing remains outside the silo bag.

The device configured as sensing unit for traditional silos (also known as static silos or metallic silos) consists of at least four metallic ring electrodes used by the grain moisture sensor, a battery pack, a power management unit, a grain moisture sensor based on electrical impedance or capacitive or microwave measurements, an oxygen sensor, a carbon dioxide sensor, an air humidity sensor, an air temperature sensor, a communication module used to communicate with the gateway, a tip in an extreme of the sensing unit, a tool to attach the unit to the cable that is firmly fixed to the silo structure and some microcontrollers used for handling the sensor data (for example, for data compression and encryption) and for processing the sensor data (for example, for sensor calibration and for pattern recognition). When the device is installed in traditional silos, it can send the data to the gateway either by using the cable that is firmly fixed to the silo structure or by using wireless communication.

The device configured as communication gateway for silo bags consists of at least four plastic rings used to hold together the different sections of the device, a battery pack, a power management unit, a communication module used to communicate with the sensing units, a communication module used to communicate either with a mobile phone network or with a satellite network, a tip in an extreme of the sensing unit, a tool for installation and sealing in the opposite extreme of the sensing unit, flexible antennas attached to the external side of the tool for installation and sealing, some fins to facilitate the insertion in the silo bag if the grains were very compacted when the silo bag was filled, an accelerometer used for the detection of a potential theft or vandalism, and a microcontroller used for handling the sensor data (for example, for data compression and encryption). When the device is installed in silo bags, it also has the particular feature that can be hidden with a cover patch thanks to the shape of its mechanical structure because only the disk of the tool for installation and sealing remains outside the silo bag.

The device configured as communication gateway for traditional silos consists of at least three plastic rings used to hold together the different sections of the device, a battery pack, a power management unit, a communication module used to communicate with the sensing units, a communication module used to communicate either with a mobile phone network or with a satellite network, a tool to attach the unit to the cable that is firmly fixed to the silo structure, a disk in an extreme of the device where flexible antennas are attached and a microcontroller used for handling the sensor data (for example, for data compression and encryption). When the device is installed in traditional silos, it can also send the data to the sensing units either by using the cable that is firmly fixed to the silo structure or by using wireless communication.

The method comprises the installation of at least two devices configured as sensing units inside each silo and the installation of at least one device configured as communication gateway inside each silo, and then the pre-processed grain data is collected and sent to a secured data center (cloud storage) using a mobile phone network or a satellite network for further data analysis and decision making.

There are many possible applications of this invention. For example some of these applications can include the ability to assess the grain moisture content inside the silo by combining the data from the moisture sensor, the air humidity sensor and the air temperature sensor.

In another application, this invention can be used to assess the biological activity inside the silo for early detection of rotting problems by combining the data from the oxygen sensor, the carbon dioxide sensor, the air humidity sensor and the air temperature sensor.

In another application, this invention can be used to detect theft or vandalism in silo bags by using the accelerometers and then alert the farmers or cooperatives, who are the owners of the silo bags.

In another application, this invention can be used to assess the proteic contents of the grains inside the silo by combining the data from the moisture sensor, the oxygen sensor, the carbon dioxide sensor, the air humidity sensor and the air temperature sensor.

In another application, this invention can be used to recognize the presence and type of fungus and insects inside all types of silos.

### BRIEF DESCRIPTION OF THE DRAWINGS

Other objects and features of the present invention will become apparent from the following detailed description considered in connection with the accompanying drawings, which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a 3D view of an assembled device with fins configured as sensing unit for silo bags;

FIG. 2 is a 3D view of a disassembled device with fins configured as sensing unit for silo bags;

FIG. 3 is a 3D view of an assembled device without fins configured as sensing unit for silo bags;

FIG. 4 is a 3D view of a disassembled device without fins configured as sensing unit for silo bags;

FIG. 5 is a 3D view of a disassembled device without fins configured as sensing unit for traditional silos;

FIG. 6 is a 3D view of a disassembled device without fins configured as communication gateway for traditional silos;

FIG. 7 is a 3D view of a disassembled device without fins configured as communication gateway for silo bags;

FIG. 8 is a view of the method to use the device in its different configurations for remote monitoring the quality and condition of grains stored in all types of silos.

FIG. 9 is a view of a photograph of a cover patch on a silo bag that hides a device under it.

### DETAILED DESCRIPTION OF PREFERRED EMBODIMENTS

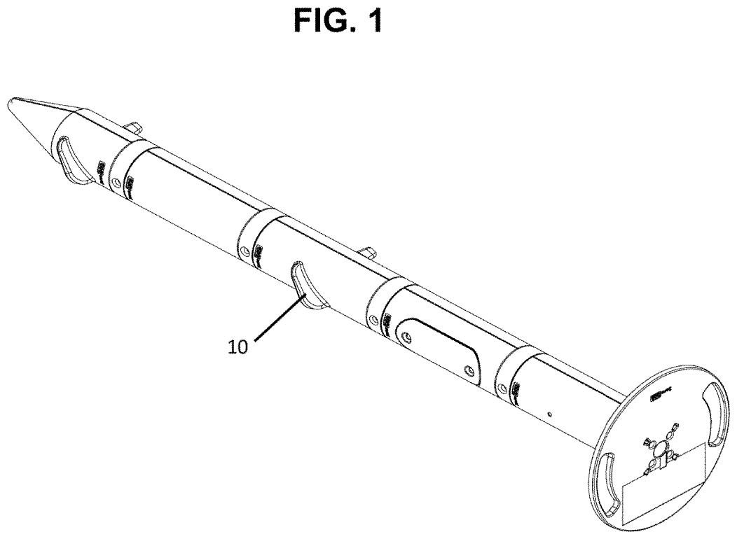

Referring to the drawings, FIG. 1 shows a 3D view of an assembled device with fins configured as sensing unit for silo bags. Thus, there is shown one of the fins 10.

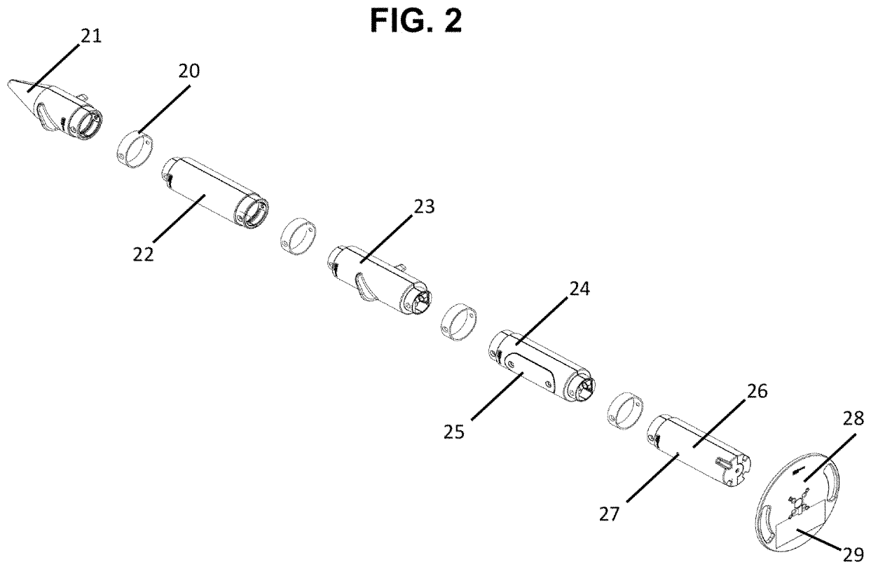

FIG. 2 shows a 3D view of a disassembled device with fins configured as sensing unit for silo bags. There is shown one of metallic ring electrodes 20 used by the grain moisture sensor, a tip 21 in an extreme of the sensing unit with its fins 10, the section 22 with the oxygen sensor, the carbon dioxide sensor, the air humidity sensor and the air temperature sensor, the section 23 with its fins 10 and with the grain moisture sensor based on electrical impedance or capacitive or microwave measurements and the microcontroller for processing the sensor data (for example, for sensor calibration and for pattern recognition), the section 24 with the battery pack and the battery cover 25, the section 26 with the power management unit, the accelerometer used for the detection of a potential theft or vandalism, the communication modules, the microcontroller used for handling the sensor data (for example, for data compression and encryption) and the hole with the light emitting diode 27, and the tool for installation and sealing 28 in the opposite extreme of the sensing unit with the flexible antenna 29 attached to the external side of it.

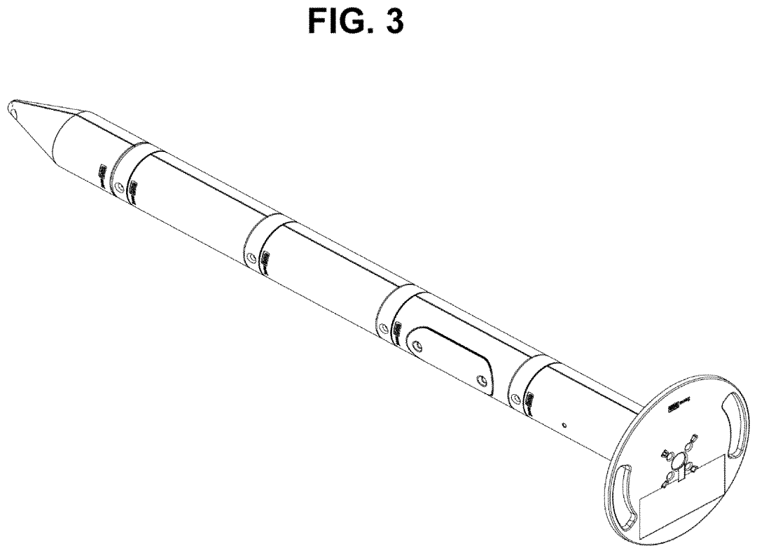

FIG. 3 shows a 3D view of an assembled device without fins configured as sensing unit for silo bags.

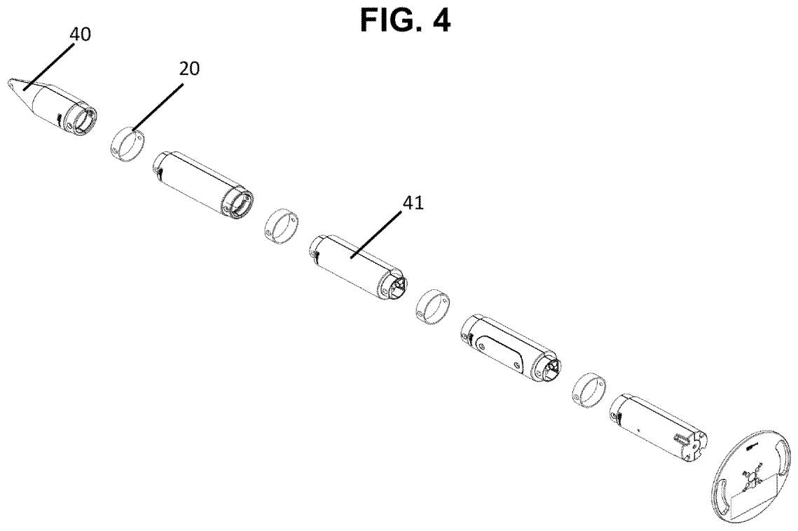

FIG. 4 shows a 3D view of a disassembled device without fins configured as sensing unit for silo bags. There is shown a tip 40 in an extreme of the sensing unit without fins, one of metallic ring electrodes 20 used by the grain moisture sensor, and the section 41 without fins and with the grain moisture sensor based on electrical impedance or capacitive or microwave measurements and the microcontroller for processing the sensor data (for example, for sensor calibration and for pattern recognition).

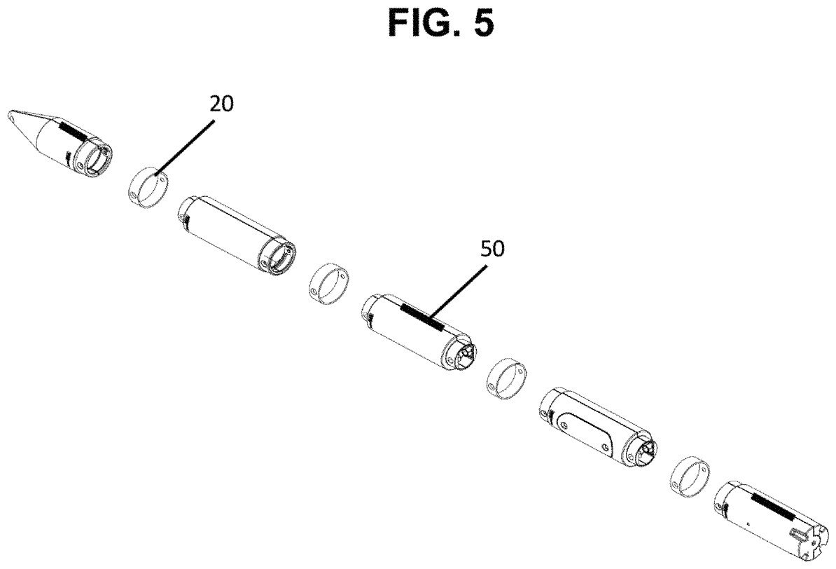

FIG. 5 shows a 3D view of a disassembled device without fins configured as sensing unit for traditional silos. There is shown one of metallic ring electrodes 20 used by the grain moisture sensor, and the tool 50 to attach the unit to the cable that is firmly fixed to the silo structure.

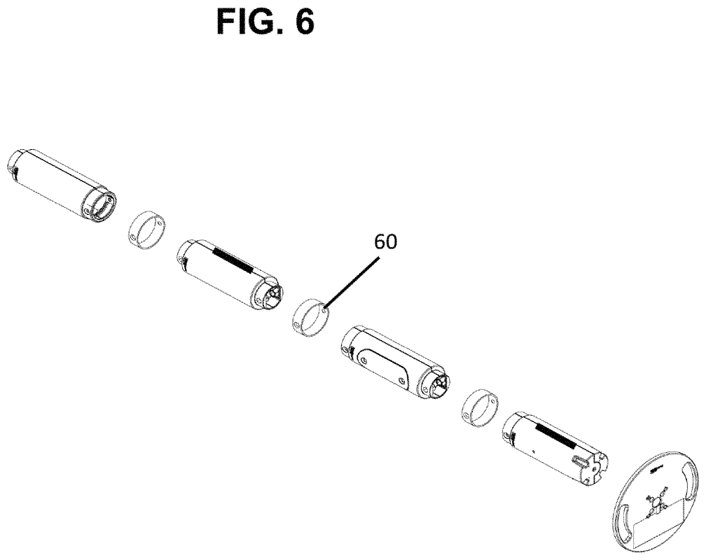

FIG. 6 shows a 3D view of a disassembled device without fins configured as communication gateway for traditional silos. There is shown one of the plastic rings 60 used to hold together the different sections of the device.

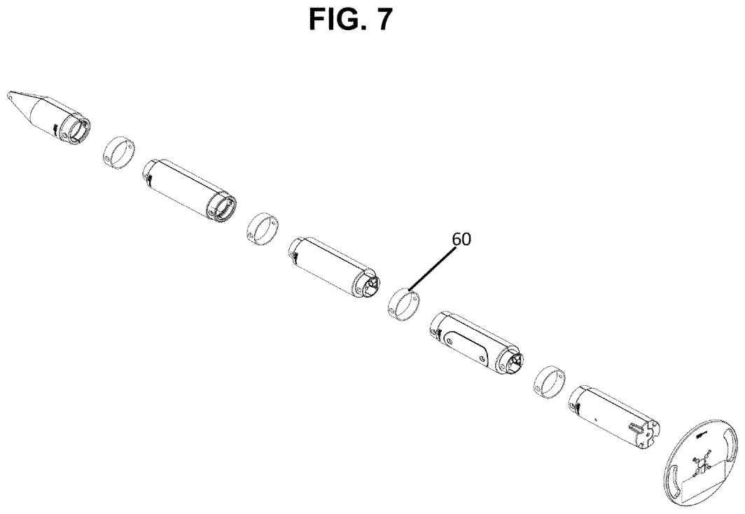

FIG. 7 shows a 3D view of a disassembled device without fins configured as communication gateway for silo bags. There is shown one of the plastic rings 60 used to hold together the different sections of the device.

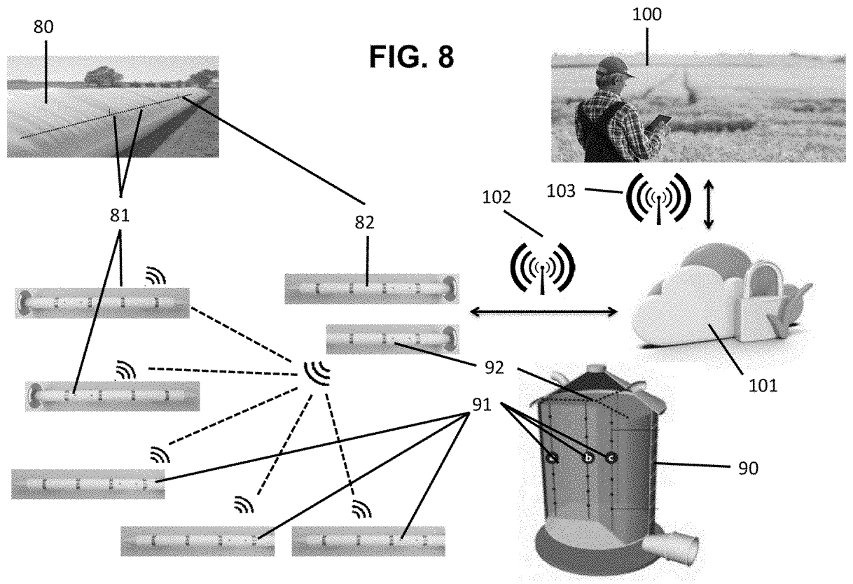

FIG. 8 shows a view of the method to use the device in its different configurations for remote monitoring the quality and condition of grains stored in all types of silos. There is shown a silo bag 80, two assembled devices without fins configured as sensing units for silo bags 81 installed inside the silo bag, an assembled device without fins configured as communication gateway for silo bags 82 installed inside the silo bag, a traditional silo 90, three assembled devices without fins configured as sensing units for traditional silos 91 installed inside the traditional silo, an assembled device without fins configured as communication gateway for traditional silos 92 installed near the roof of the traditional silo, the farmers 100 or cooperatives that receive the data via WiFi or Bluetooth 103, and the secured data center (cloud storage) 101 that receives the data via a mobile phone network or a satellite network 102.

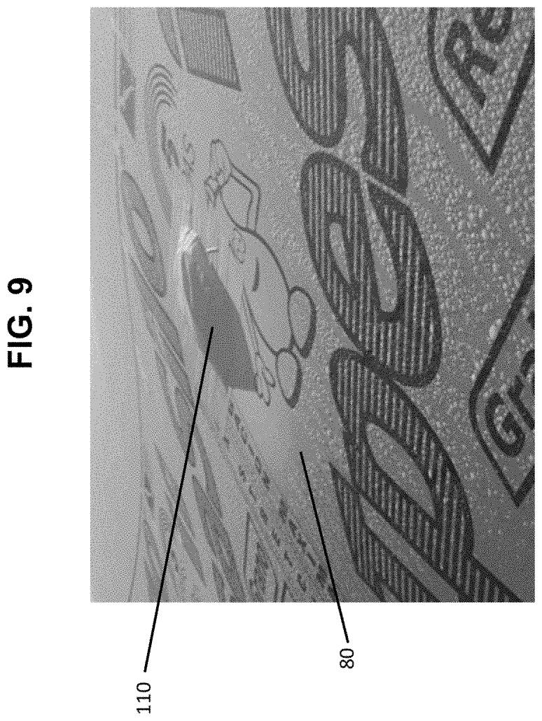

FIG. 9 shows a view of a photograph of a cover patch 110 on a silo bag 80 that hides a device under it to avoid the robbery of the devices.

Accordingly, while at least one embodiment of the present invention have been shown and described, it is to be understood that many changes and modifications may be made thereunto without departing from the spirit and scope of the invention as defined in the appended claims.

What is claimed is:

1. An apparatus for remote monitoring the quality and condition of grains stored in all types of silos that can be configured either as a sensing unit or as a communication gateway.

2. The apparatus of claim 1 when configured as sensing unit comprises:
a housing;
a grain moisture sensor board;
a gas sensor board, including an oxygen sensor and a carbon dioxide sensor;
a communication board, including an antenna, an accelerometer, and air temperature and an air humidity sensor; and
a battery pack.

3. The apparatus of claim 1 when configured as communication gateway comprises:
a housing;
a communication board, including two antennas, an accelerometer, and air temperature and an air humidity sensor; and
a battery pack.

4. The apparatus of claim 1 can be used for remote monitoring of soil moisture.
